(19) United States
(12) Patent Application Publication
(10) Pub. No.: US 2021/0267614 A1
(43) Pub. Date: Sep. 2, 2021

(54) MULTI-PILLAR PIEZOELECTRIC STACK ULTRASOUND TRANSDUCER AND METHODS FOR USING SAME

(71) Applicant: North Carolina State University, Raleigh, NC (US)

(72) Inventors: Xiaoning Jiang, Cary, NC (US); Howuk Kim, Raleigh, NC (US)

(21) Appl. No.: 17/198,926

(22) Filed: Mar. 11, 2021

Related U.S. Application Data
(63) Continuation-in-part of application No. 17/016,304, filed on Sep. 9, 2020.
Application No. 17/016,304 is a continuation-in-part of application No. 16/317,983, filed on Jan. 15, 2019.
(60) Provisional application No. 62/362,687, filed on Jul. 15, 2016.
(60) Provisional application No. 62/897,759, filed on Sep. 9, 2019.

(51) Int. Cl. A61B 17/22

(52) U.S. Cl. CPC A61B 17/2202; A61B 2017/00402

(57) ABSTRACT

A multi-pillar piezoelectric stack (MPPS) ultrasound transducer includes N pillars, each formed of a stack of M piezoelectric elements, N and M being integers of at least two. The ultrasound transducer further includes a bonding layer between each pair of the M piezoelectric elements. The pillars are laterally spaced from each other to form an inter-pillar gap. The transducer further includes at least one electrical interconnect for connecting the ultrasound transducer to a signal source. Through the MPPS design, the therapeutic range and the transducer sensitivity are increased over the conventional single pillar piezoelectric stack (SPPS) transducer design.

### RELATED APPLICATIONS

This application is a continuation-in-part of U.S. patent application Ser. No. 17/016,304 filed Sep. 9, 2020, which is a continuation-in-part of U.S. patent application Ser. No. 16/317,983 filed Jan. 15, 2019, which is a national stage application under 35 U.S.C. § 371 of PCT Application Number PCT/US2017/042372, filed Jul. 17, 2017, which claims the benefit of U.S. Provisional Patent Application Ser. No. 62/362,687, filed Jul. 15, 2016. U.S. patent application Ser. No. 17/016,304 further claims the benefit of U.S. Provisional Patent Application Ser. No. 62/897,759, filed Sep. 9, 2019. The disclosures of each of the aforementioned applications is incorporated herein by reference in its entirety.

### GOVERNMENT INTEREST

This invention was made with government support under Grant Number R01 HL141967 awarded by the National Institutes of Health. The government has certain rights in the invention.

### TECHNICAL FIELD

The subject matter described herein relates to ultrasound transducers. More particularly, the subject matter described herein relates to a multi-pillar piezoelectric stack ultrasound transducer and methods for using same.

### BACKGROUND

Ultrasound transducers can be miniaturized to be insertable within blood vessels to deliver ultrasound energy from within the blood vessels. Desired characteristics of such transducers are the ability to deliver a focused beam of ultrasound energy at increased distances from the transducer aperture with minimal effects on tissue surrounding the target of the ultrasound energy, which may be a blood clot or other target.

Piezoelectric materials are increasingly being used for ultrasound transducers because of their fast response time (high frequency operation), cost-effectiveness, and processability of material to a miniature size. However, single-pillar piezoelectric stack ultrasound transducers do not operate efficiently at frequency bands greater than or equal to 1 MHz and may not deliver sufficient acoustic power at distances greater than 1 mm from the transducer aperture. In addition, single-pillar piezoelectric stack ultrasound transducers may deliver unwanted acoustic radiation in lateral directions, which may adversely affect surrounding tissues and/or vessel walls.

As a result, there exists a need for an improved ultrasound transducer that avoids at least some of the difficulties associated with conventional ultrasound traducers designed for intravascular therapy.

### SUMMARY

A multi-pillar piezoelectric stack ultrasound transducer includes N pillars, each formed of a stack of M piezoelectric elements, N and M being integers of at least two. The transducer further includes a bonding layer between each pair of the M piezoelectric elements. The pillars are laterally spaced from each other to form an inter-pillar gap. The transducer further includes at least one electrical interconnect for connecting the ultrasound transducer to a signal source.

A system for delivering ultrasound energy from within a body of a subject includes a multi-pillar piezoelectric stack ultrasound transducer including N pillars, each formed of stacks of M piezoelectric elements, N and M being integers of at least two. The ultrasound transducer further includes a bonding layer between each pair of the M piezoelectric elements. The N pillars are laterally spaced from each other to form an inter-pillar gap. The ultrasound transducer further includes at least one electrical interconnect for connecting the ultrasound transducer to a signal source. The system further includes a catheter, where the ultrasound transducer is deployable from within the catheter.

A method for delivering ultrasound energy from within a body of a subject includes inserting, within a body of a subject, a multi-pillar piezoelectric stack ultrasound transducer including: N pillars, each formed of stacks of M piezoelectric elements, N and M being integers of at least two; a bonding layer between each pair of the M piezoelectric elements, wherein the pillars are laterally spaced from each other to form an inter-pillar gap; and at least one electrical interconnect for connecting the ultrasound transducer to a signal source. The method further includes applying an electrical signal to the multi-pillar piezoelectric stack ultrasound transducer via the at least one electrical interconnect, which causes the pillars to vibrate and deliver ultrasound energy from within the body of the subject.

The subject matter described herein may be implemented in hardware, software, firmware, or any combination thereof. As such, the terms “function” “node” or “module” as used herein refer to hardware, which may also include software and/or firmware components, for implementing the feature being described. In one exemplary implementation, the subject matter described herein may be implemented using a computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

FIG. 1 is a schematic diagram of nanodroplet mediated ultrasound thrombolysis inside of a blood vessel;

FIG. 2A is a schematic diagram illustrating mechanical structures of a single pillar piezoelectric stack (SPPS) transducer;

FIG. 2B is a schematic diagram of a multi-pillar piezoelectric stack (MPPS) transducer where the vertical arrows indicate thickness vibration mode and the laterally-extending arrows indicate lateral vibration mode;

FIG. 3 illustrates the finite element (FE) model and boundary conditions for a simulation of operation of the MPPS transducer;

FIGS. 4A1-4A3 illustrate an exemplary fabrication procedure and structure for the MPPS transducer;

FIG. 4B is an image of the fabricated transducer;

FIG. 5 is a schematic diagram of a test setup for testing the MPPS transducer;

FIG. 6 illustrates an in vitro test setup involving the intravascular US dynamic flow model for the demonstration of thrombolysis;

FIGS. 7A-7C illustrate simulation results of the SPPS and MPPS transducers; FIG. 7A illustrates electric impedance responses; acoustic pressure fields by the SPPS (FIG. 7B) and by the MPPS transducer (FIG. 7C);

FIGS. 8A-8C illustrate experimental results of the fabricated MPPS transducer; (FIG. 8A) electric impedance responses, (FIG. 8B) acoustic pressure fields and (FIG. 8C) the sensitivity of the pressure output at the focal spot and the mechanical index;

FIG. 9 illustrates representative images of the demonstration of nanodroplet-mediated thrombolysis for 30 minutes of sonication, where the white arrow in each image indicates the vertical position of the transucer;

FIG. 10 is a graph illustrating a comparison of thrombolytic rate under four treatment groups (without flow): 1) control, 2) ultrasound only, 3) ultrasound with MB, and 4) ultrasound with ND (n=3);

FIGS. 11A-11C illustrate the influence of ND concentrations from 0 to 10⁹ ND/mL (FIG. 11A) and the blood clot prior to (FIG. 11B) and after (FIG. 11C) the treatment at 10⁹ ND/mL, where the number of tests is three for each test group;

FIG. 12 is a flow chart illustrating a method for using an MPPS transducer for intravascular therapy; and

FIGS. 13A-13D illustrate results of a study of the ND cavitation effect using the MPPS transducer; (FIG. 13A) acoustic pressure output in time domain under 80 Vpp, (FIG. 13B) frequency spectrum with respect to input voltage level, and quantifications of (FIG. 13C) stable and (FIG. 13D) inertial cavitation, where the inset (rectangular box) figure in FIG. 13A represents the nonlinearity of the wave signal and the circles in FIG. 13B indicate super-harmonic terms of the wave signal.

### DETAILED DESCRIPTION

The subject matter described herein includes a nanodroplet (ND)-mediated intravascular ultrasound (US) transducer for deep vein thrombosis treatments. The US device, having an efficient forward directivity of the acoustic beam, is capable of expediting the clot dissolution rate by activating cavitation of NDs injected onto a thrombus. Methods: We designed and prototyped a multi-pillar piezoelectric stack (MPPS) transducer composed of four piezoelectric pillars. Each pillar was made of five layers of lead zirconate titanate (PZT) plates having a dimension of 0.85×0.85×0.2 mm³. The transducer was characterized by measuring the electric impedance and acoustic pressure, compared to simulation results. Next, in-vitro tests were conducted in a blood flow mimicking system using the transducer equipped with an ND injecting tube. Results: The miniaturized transducer, having an aperture size of 2.8 mm, provided a high mechanical index of 1.52 and a relatively wide focal zone of 3.4 mm at 80 Vₚₚ, 0.96 MHz electric input. The mass-reduction rate of the proposed method (NDs+US) was assessed to be 2.3 and 2.5%/min with and without the flow model, respectively. The rate was higher than that (0.7-1.5%/min) of other intravascular ultrasound modalities using micron-sized bubble agents. Conclusion: The ND-mediated intravascular sonothrombolysis using MPPS transducers was demonstrated with an unprecedented lysis rate, which may offer a new clinical option for DVT treatments. Significance: The MPPS transducer generated a high acoustic pressure (˜3.1 MPa) at a distance of approximately 2.2 wavelengths from the small aperture, providing synergistic efficacy with nanodroplets for thrombolysis without thrombolytic agents.

### INTRODUCTION

Deep vein thrombosis (DVT), the formation of a thrombus in the deep venous system, may be induced by certain factors such as immobility (e.g., prolonged bed rest, obesity, and surgery) and hypercoagulation caused by smoking, injury to veins, cancer, genetic issue, and leg fracture [1]. Common symptoms of DVT include leg pain, swelling, and skin discoloration [2]. Furthermore, thrombus in legs can result in serious conditions when a piece of the blood clot travels through the circulation system and lodges in one of the pulmonary arteries, resulting in pulmonary embolism [3]. Reduced amount of blood flow into the lung, due to the blockage, may also decrease the amount of oxygen absorbed by the lung, causing a life-threatening condition with a high sudden death rate (>25%) [4]. It is, therefore, crucial to treat DVT appropriately and promptly to minimize potential complications.

DVT is mostly treated through medications, such as anticoagulants, that make it hard for blood to clot [5]. Anticoagulants prevent blood clots from getting bigger and traveling through the bloodstream. However, some patients cannot take anticoagulants because of bleeding risk [6]. In addition, the treatment requires a relatively long time (e.g., at least three months) for blood clots to be dissolved naturally [7]. Meanwhile, mechanical treatment can be considered for patients who cannot have medication treatments. For instance, vena cava filters, placed via minor surgery, prevent thrombus from moving to the heart and lung [5], [8], although such mechanical filters still have potential complications, such as perforation with retroperitoneal bleeds, embolization, and filter fracture [9]. Thrombolysis using the focused US has been recently introduced by some researchers [10]-[12]. The focused US is capable of dissolving thrombus noninvasively without surgery [13], whereas the US must be precisely directed to the target region so as not to damage unwanted surrounding tissues [14]. These current techniques still do not provide an optimal clinical solution for DVT treatments.

Interstitial therapeutic US devices have recently shown clinical potential considering overall clinical aspects, such as safety, cost, treatment time burden, and effectiveness-to-risk ratio. In contrast to typical noninvasive focused US methods, intravascular transducers, due to their relatively small geometric dimension, allow the localized sonification of a target region while suppressing excessive exposure of surrounding tissue and organs [15]-[18]. Direct interaction with the target enables the intravascular device to achieve the clinical goal with a relatively low electric power (<20 W) [15]-[18]. Furthermore, the interstitial transducer can precisely deliver a sufficiently high acoustic pressure over a target region without damaging unwanted tissues [15], [18], [19]. Meanwhile, some researchers investigated intravascular US transducers, capable of performing in a small vascular (2-5 mm), for a variety of therapeutic purposes, such as tissue ablation [19], [20], drug delivery [21], [22], and thrombolysis [23], [24]. In 2014, the FDA approved a side-looking, minimally invasive endovascular therapeutic device (EkoSonic™ Endovascular System, Boston Scientific, Marlborough, Mass.) for use in treating pulmonary embolism [25]. The therapeutic efficacy of thrombolysis has been demonstrated using the device, along with microbubble injection [26]. This FDA-approved technique has demonstrated safe and improved permeation of recombinant tissue plasminogen activator (rt-PA) by low-power ultrasound. However, some randomized clinical trials show that this low-power ultrasound effect is not significant, even showing no difference between localized rt-PA delivery alone and ultrasound-assisted delivery [27]. Higher power output may solve this problem but its side-viewing design that directly aims vessel lumen hinders applying this easy solution. J. Kim et al. [23] suggested a microbubble (MB)-mediated intravascular sonothrombolysis technique, using a miniaturized forward-viewing US transducer that has a relatively high yet spatially confined acoustic pressure output (˜2 MPa in the peak-to-peak level) with the aid of a multilayered piezoelectric stack and a concave lens. The therapeutic efficiency of the forward-viewing device, combined with medication (i.e., rt-PA), was further demonstrated in [28]. B. Zhang et al. [29] utilized the cavitation of magnetic MBs concentrated around the target clot by using a forward-viewing transducer. However, existing intravascular US transducers have a relatively short focal distance (<1-1.5 mm) due to their small aperture size and relatively low operation frequency (<0.7 MHz). The short spatial coverage in an axial direction is disadvantageous to cause cavitation effects of MBs in a sufficient target volume, thus limiting treatment efficiency (<30-50% mass reduction for 30 min in-vitro).

Microbubbles (i.e., micron-sized, lipid-shelled gas bubbles) are known to increase the rate of thrombolysis, in combination with sonication, over a certain mechanical index (MI) level (i.e., MI>0.3 for inertial cavitation) [30]. However, MBs have a relatively short lifetime in-vivo, which restricts effective therapeutic time during US treatments [31]. Moreover, due to their relatively large size (approximately 2 μm on average), MBs may remain confined on tissue surface and not penetrate the target region. In contrast, nanodroplets (ND), composed of liquid (condensed) perfluorocarbons with a smaller diameter (<300 nm on average), exhibit effective permeability to a target tissue [32]. Furthermore, NDs remain viable for a relatively longer time than MB agents in blood circulation [33]. Despite the potential advantage of NDs, applications incorporated with intravascular US transducers are hardly found in the literature due to the insufficient acoustic pressure and the limited focal range of existing intravascular devices [27]-[29].

The goals of this study are to (1) develop a customized intravascular US transducer that overcomes the limitation of current intravascular transducers by transmitting sufficient acoustic pressure over a long distance (>2 wavelengths) under a sub-megahertz operation condition, and (2) to evaluate the therapeutic efficacy of ND-mediated thrombolysis combined with the new device under static and dynamic flow models, respectively. It was hypothesized that (1) nano-sized droplets can more effectively permeate a deep region of a target clot than other micron-size bubbles, and (2) the acoustic droplet vaporization and cavitation of NDs with more proximity to the target center can be activated by delivering sufficiently high acoustic energy generated by the developed transducer.

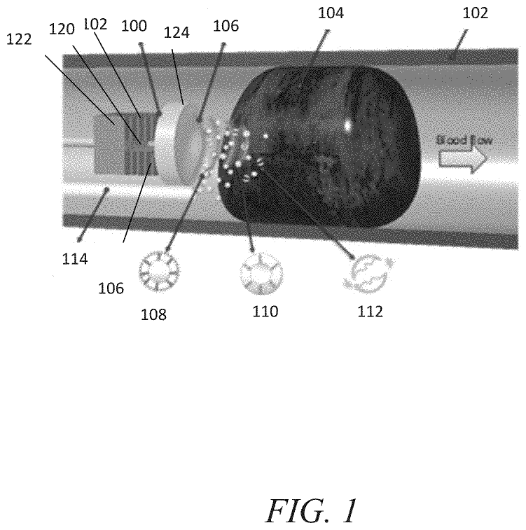

FIG. 1 presents a schematic view of the ND-mediated intravascular US thrombolysis. In FIG. 1, an ultrasound transducer 100 is configured to be deployed within a body of a subject, such as within a blood vessel 102 to lyse a blood clot 104. In particular, ultrasound transducer 100 generates ultrasound waves 106 which cause phase change nanodroplets that penetrate blood clot 104 to change phase, cavitate, and burst to lyse blood clot 104 from within. In FIG. 1, nanodroplet 108 is undergoing the phase change from a nanodroplet to a microbubble. Microbubble 110 experiences stable cavitation caused by ultrasound waves 106. Microbubble 112 experiences inertial cavitation and bursts. The nanodroplets are deployed in blood vessel 102 using an injection tube 114.

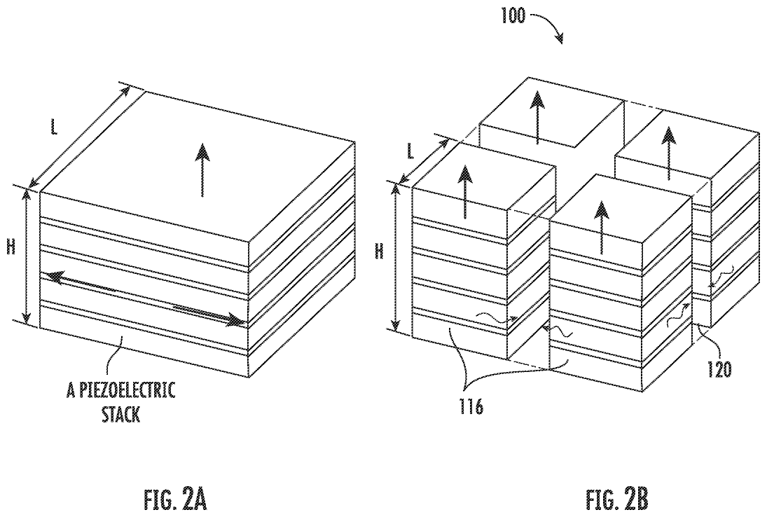

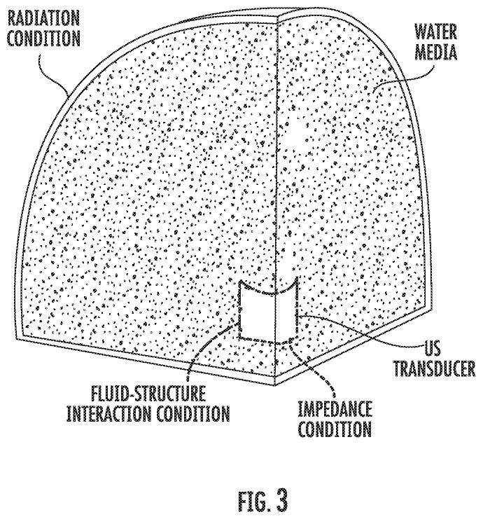

Structurally, as illustrated in FIG. 2B, transducer 100 includes N pillars 116 of M stacked piezoelectric elements 118, where N and M are each integers of at least 2. In the illustrated example N=4 and M=5. Pillars 116 are axially spaced from each other by inter-pillar gaps 120. Gaps 120 may be air gaps or may be filled with a material, such as a polymer, designed to limit lateral vibrations of pillars 116. Transducer 100 further includes a backing layer 122 from which pillars 116 extend axially. Transducer 100 further includes an acoustic impedance matching layer 124 for acoustic impedance matching between pillars 116 and an operating medium of ultrasound transducer 100. In one example, acoustic impedance matching layer 124 may comprise an acoustic lens with a concave axially facing outer surface for focusing acoustic energy. In another example, acoustic impedance matching layer 124 may comprise a flat aperture (a layer with a flat axially facing outer surface) for acoustic impedance matching and delivering a substantially flat acoustic wavefront.

In the study described herein, piezoelectric elements 118 in the prototype transducer were made of PZT-4 material. In an alternate implementation, piezoelectric elements 118 may be made of soft piezoelectric materials, such as PZT-5H, PZT-5A, lead magnesium niobite-lead titanate (PMN-PT), etc. or hard piezoelectric materials other than PZT-4, such as PZT-2, PZT-8, etc. In another example, piezoelectric elements 118 may be made of a lead-free piezoelectric material. The type of material for piezoelectric elements 118 depends on the application. For example, soft piezoelectric materials can be used in generating short-time, high-amplitude acoustic pulses with a relatively low electrical input, and hard piezoelectric materials can be used for transmitting acoustic power in a continuous wave signal.

### Materials and Methods

### Transducer Design

The intravascular US transducer needs to transmit a high acoustic pressure output, causing a sufficient MI (>0.3) for agent-assisted inertial cavitation [30], onto a relatively far distance (>3 mm) from the small aperture to generate effective cavitation of ND. MI level induced by US wave is defined as follows [34]:

MI=P̅/\sqrt{f} (1)

where P̅ is the negative pressure level in MPa, and f is the frequency in MHz. (1) indicates that a relatively low frequency (e.g., <1 MHz) is advantageous to achieve a high MI. However, it is relatively difficult for sub-megahertz transducers to achieve a long focal distance since the Fresnel zone depends on wavelength as follows [35]:

Fresnel zone=r²/λ (2)

where r is the radius of aperture, and λ is the wavelength. For example, the far-field of a 1 MHz transducer having a diameter of 2 mm would begin from the distance of the half wavelength. As such, novel design approaches are needed for sub-megahertz transducers to extend the forward directivity of the acoustic beam to a relatively far distance (>2λ).

Sub-megahertz transducers require a relatively thick (>1 mm) dimension of the active material. The electrical impedance of the thick piezoelectric plate becomes relatively high due to the low capacitance, requiring a high input voltage to drive the transducer. A multilayered design for the piezoelectric stack was thus adopted for the ultrasound transducer to reduce the impedance level at the driving frequency. For the active layer, PZT-4 material was used because of its high mechanical quality factor and electric robustness [36]. Five active layers (200 μm-thick each) were stacked together to decrease electric impedance and intensify acoustic power with the extensional mode. The overall thickness of active layers (i.e., 1 mm) was smaller than the lateral dimension of the transducer (2 mm); hence, the lateral vibration mode can be more predominant at the first resonance frequency. For the suppression of the lateral vibration mode, the concept of a 1-3 composite structure (1-D connection of piezoelectric element and 3-D connection of polymer matrix) was adopted for the transducer design. Notably, our multi-pillar piezoelectric stack (MPPS) design is different from simply stacking up conventional 1-3 composite layers [37], [38]; 1) the MPPS design comprises relatively large-cross-sectional-area pillar (>λ) compared to conventional 1-3 composite designs (<0.2λ). This makes MPPS oscillation separated from polymer matrix oscillation, whereas a fine periodic dimension is crucial for the homogenized, in-phase oscillation of conventional 1-3 composites. 2) a fine-periodic structure of 1-3 composite is considered as an effective single-phase medium typically with 50-70% stiffness of piezoelectric ceramics. The lowered stiffness makes the single pillar stacked-layer transducer (FIG. 2A) vulnerable to damped oscillation effect caused by intermediate bonding layers compared to the same structure of PZT ceramic pillars as shown in MPPS design (FIG. 2B). In FIG. 2B, MPPS transducer 100 includes four pillars 116 that are separated by inter-pillar gap 120, which in one example is filled with polydimethylsiloxane (PDMS), to realize pure extensional vibration suppressing both mode coupling and bonding layers effects. In another example, inter-pillar gap 120 may be filled with air or other gas or an epoxy material. Next, a concave metallic lens (see FIG. 1) with a radius curvature of 2 mm was integrated on top of MPPS transducer 100 to focus the acoustic pressure output. Lastly, backing layer 122 (see FIG. 1) was applied at the rear side of the MPPS for the effective transmission of the acoustic wave to the forward direction.

Another difference between the MPPS design in FIG. 2B and the SPPS design in FIG. 2A is that in the SPPS design, the pillar is greater in lateral dimensions (labeled L in FIGS. 2A and 2B) than in the axial direction (labeled H in FIGS. 2A and 2B); whereas in the MPPS design, each pillar 116 is greater in axial than in either lateral dimension (length or width, whether equal or unequal). Having an axial length that is greater than lateral dimensions enables the MPPS pillars 116 to produce acoustic energy beams that are more focused in the axial direction and at greater distances than the SPPS design. The overall height and thickness of MPPS transducer 100, as well as the thickness of each piezoelectric element 118, may be selected based on the operational frequency range of MPPS transducer 100. Similarly, lateral dimensions of MPPS transducer 100 may be selected based on the geometry of the target environment. For example, for intra-vascular applications, the lateral dimension of MPPS transducer 100 may be selected such that MPPS transducer 100 can be deployed within a blood vessel. Another feature of the MPPS design in FIG. 2B is the lateral separation between pillars 116, which does not exist in the SPPS design, as there is only one pillar.

### Numerical Simulation

Numerical simulations were conducted using ANSYS (Rel. 17.1, ANSYS, Inc., Canonsburg, Pa.), a commercial finite element (FE) analysis software, to predict the performance of the designed transducer. Table I lists the material properties of individual parts in the transducer. FIG. 3 presents the boundary conditions of the FE model. Fluid-structure interaction (FSI) condition was applied to the interface between the transducer model and the water media, which transforms the mechanical vibration of the transducer into acoustic pressure in water. Meanwhile, acoustic radiation condition was used at the outer surfaces of the water media to restrict reflections at the boundaries. The acoustic impedance of 500 Rayls was applied at the rear surface of the transducer to simulate air-backing [40]. The FE analysis was performed for a single-pillar piezoelectric stack (SPPS) transducer (FIG. 2A) to confirm the effectiveness of the MPPS design (FIG. 2B).

TABLE I
MATERIAL PROPERTIES OF THE MPPS TRANSDUCER
[19], [39].

| | Properties | Value | | Properties | Value |
|---|---|---|---|---|---|
| PZT-4 | ρ (kg/m³) | 7,500 | Aluminum | ρ (kg/m³) | 2,700 |
| | S₁₁^{E} (×10⁻¹² | 12.3 | | Y (GPa) | 70.0 |
| | Pa⁻¹) | | | | |
| | S₃₃^{E} (×10⁻¹² | 15.5 | | ν | 0.33 |
| | Pa⁻¹) | | | | |
| | S₃₁^{E} (×10⁻¹² | −5.31 | E-solder | ρ (kg/m³) | 2600 |
| | Pa⁻¹) | | | | |
| | S₁₅^{E} (×10⁻¹² | 39.0 | | Y (GPa) | 5.8 |
| | Pa⁻¹) | | | | |
| | e₃₁ (C/m²) | −5.2 | | ν | 0.38 |
| | e₃₃ (C/m²) | 15.1 | PDMS | ρ (kg/m³) | 1,030 |
| | ∈₁₁^{S}/∈₀ | 762 | | Y (GPa) | 1.32 × 10⁻⁴ |
| | ∈₃₃^{S}/∈₀ | 663 | | ν | 0.49 |

Sₓₓ^{E}: compliance under free electric field, eₓₓ: piezoelectric coefficient, ∈ₓₓ^{S}/∈₀: dielectric constant under free strain, Y: Young's modulus, ρ: density, and v: Poisson's ratio.

### Transducer Fabrication and Characterization

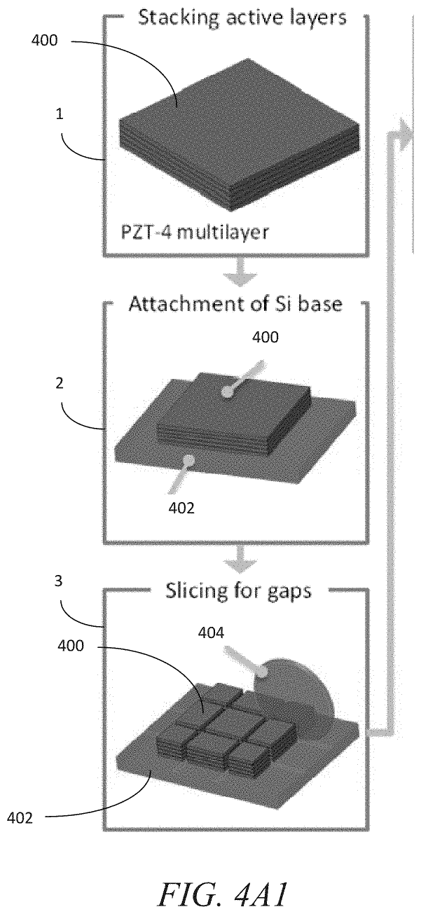

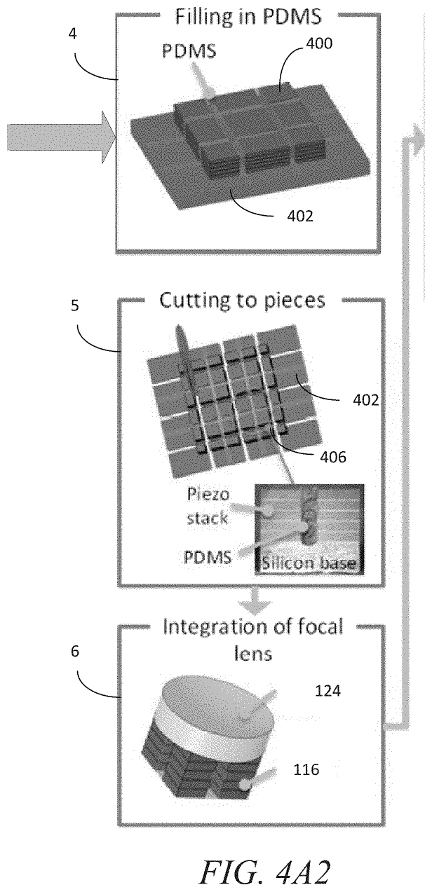

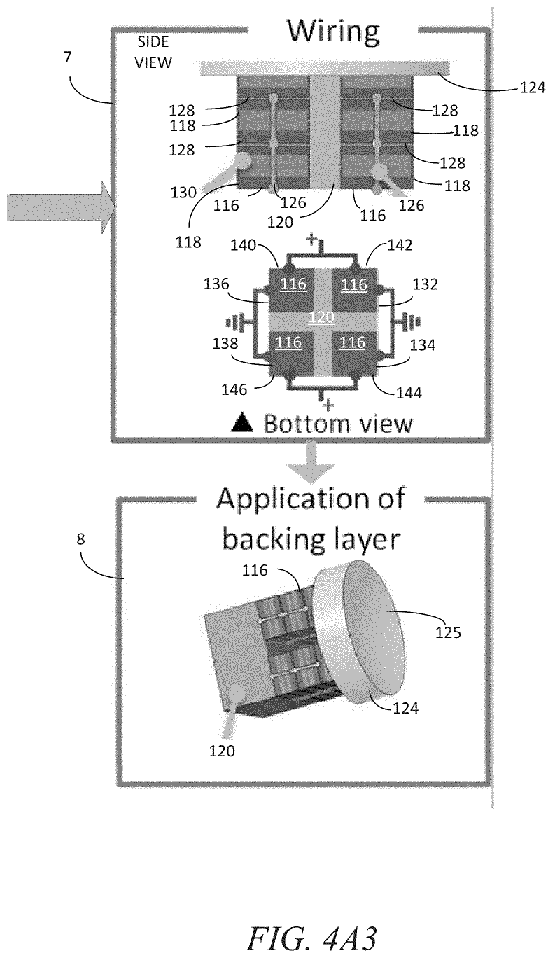

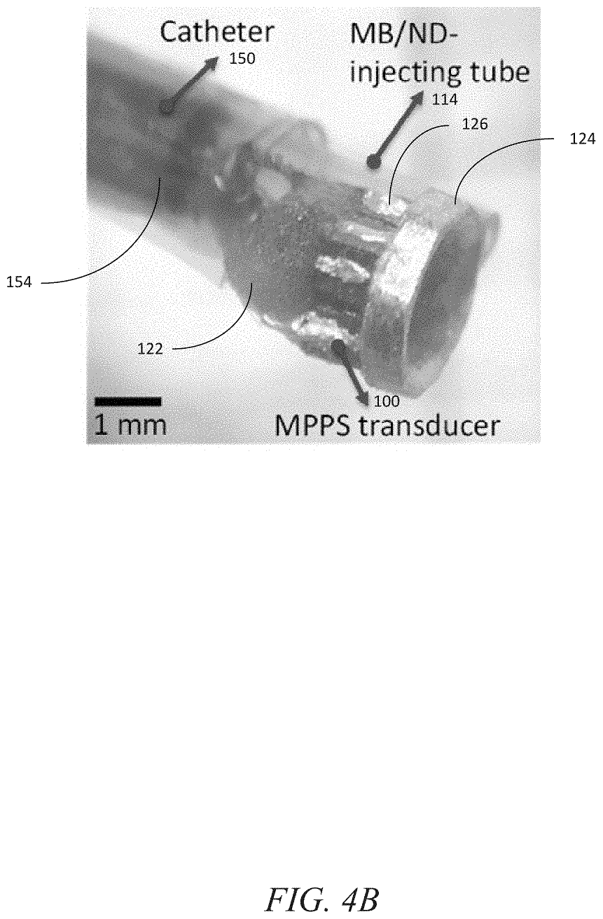

FIGS. 4A1-4A3 illustrate a method for fabricating MPPS transducer 100. Each active layer (i.e., PZT-4) was lapped into the thickness of 200 μm, followed by the deposition of the electrodes with Au/Cr (200/10 nm). Each active layer was stacked together, using conductive silver epoxy (E-Solder 3022, Von Roll Inc., Cleveland, Ohio) as an intermediate bonding layer (approximately 20 μm). The piezoelectric stack was attached to a silicon wafer, using a wax-resin to hold the specimen for the following dicing process. The stack was partially sliced with a kerf width of 300 μm (DISCO 322, DISCO Hi-Tec America, Inc., San Jose, Calif.). The gap was filled with PDMS (Sylgard™ 184, Dow Corning, Midland, Mich.), and the stack was diced to achieve a sectional dimension of 2×2 mm², making it a 1-3 composite structure. After integrating a metallic concave lens on top of the MPPS, the silicon substrate was removed from the sample. The electrodes of the MPPS were connected with a coaxial cable (5381-006, AWG 38, Hitachi Cable America Inc., Manchester, N.H.) after isolating unneeded electrodes, using Parylene-C sheets. Meanwhile, air-backing was fabricated by making a composite of air microbubbles (Blatek Inc., State College, Pa.) and epoxy (Epoteke 301, Epoxy Tech. Inc., San Jose, Calif.) with a volume ratio of 3:1. The mixture was applied at the rear side of the MPPS, with a thickness of about 1 mm. Finally, a parylene-C layer was deposited on the transducer surface to make it waterproof by using a Parylene coater (SCS Labcoter®, PDS 2010, SCS, Indianapolis, Ind.).

The exemplary method for manufacturing a multi-pillar piezoelectric stack (MPPS) ultrasound transducer begins in FIG. 4A1, step 1, where the fabrication process includes forming a stack 400 of active piezoelectric layers, which in the examples described herein are layers of lead zirconate titanate. In step 2, the process includes attaching a silicon base 402 to stack 400. In step 3, stack 400 is cross-cut in orthogonal directions using a dicing saw 404 to create separate stacks of piezoelectric layers with axially extending gaps between the stacks.

Referring to FIG. 4A2, in step 4, the process includes filling the gaps between the layers with a polymer material, which in the examples described herein is polydimethylsiloxane (PDMS). In step 5, the process includes cross cutting the stacks formed in step 3 into groups of four equally sized layered pillars 116 of stacked piezoelectric elements 118, where each group of four pillars 116 may be used as a transmit head for an ultrasound transducer. In step 6, the process includes removing the silicon base and attaching acoustic impedance matching layer 124 to each group of four pillars 116. In the examples in FIGS. 4A1-4A3, acoustic impedance matching layer 124 comprises an acoustic lens with a concave axially-facing outer surface 125. In an alternate implementation, acoustic matching layer 124 may comprise a substantially flat acoustic aperture (i.e., a metallic cylinder with a substantially flat axially-facing outer surface). The acoustic impedance of acoustic impedance matching layer 124 may be selected to be between that of piezoelectric elements 118 and the surrounding medium in the target application. For example, if the acoustic impedance of piezoelectric elements 118 is 30 Mrayl/m² and the acoustic impedance of the surrounding medium during operation is 1.6 Mrayl/m² then the acoustic impedance of acoustic impedance matching layer 124 may be selected to be 15.8 Mrayl/m², which is the average of the acoustic impedances of piezoelectric elements 118 and the surrounding medium during operation.

Referring to FIG. 4A3, the process includes, in step 7, interconnecting bonding layers 128 between stacked piezoelectric elements 118 using electrical interconnects 126. In the illustrated example, electrical interconnects 126 extend axially along lateral faces of each pillar 116 as illustrated by the side view. Electrical interconnects 126 may be formed of any conductive material capable of conducting an electrical signal to or from piezoelectric elements 118. In one example, electrical interconnects 126 are formed using solder traces deposited on the sidewalls of pillars 116. Electrical interconnects 126 connect to nonadjacent bonding layers 128 between piezoelectric elements 118. As illustrated by the bottom view, electrical interconnects 126 on lateral faces 132, 134, 136, and 138 are connected to ground, and electrical interconnects 126 on lateral faces 140, 142, 144, and 146 are connected to a signal source. It should also be noted that electrical interconnects 126 on orthogonal lateral faces of each pillar 116 are connected to different bonding layers 128 so that a potential difference can be developed between axially adjacent bonding layers. For example, in the side view, the electrical interconnects 126 are connected to the bonding layers between the second and third piezoelectric elements 118 and the fourth and fifth piezoelectric elements 118. The electrical interconnects on lateral faces of pillars 116 that are orthogonal to those shown in the side view are connected to the bonding layers between the first and second piezoelectric elements 118 and the third and fourth piezoelectric elements 118. It should also be noted that on any given lateral face, the bonding layers that are not connected to the electrical interconnect 126 on that face may be coated with a non-conductive layer, such as a parylene-C layer 130. In step 8, base or backing layer 122 is connected to the ends of pillars 116 that are opposite acoustic impedance matching layer 124. As described above, in one example, backing layer 122 is made of an epoxy resin material with internal air bubbles or voids. However, any suitable material with relatively low acoustic impedance (<<1 Mrayl/m²) may be used. In yet another alternate implementation, backing layer 122 may comprise an enclosure that defines an air cavity, a composite material with internal air bubbles, and a polymer.

FIG. 4B is an image of a prototype of MPPS ultrasound transducer 100 deployable from within a catheter. In FIG. 4B, MPPS ultrasound transducer 100 is deployable from within a catheter 150.

Microbubble/nanodroplet injecting tube 114 may also be deployable from within catheter 150. In the illustrated example, microbubble/nanodroplet injecting tube 114 is positioned off axis from MPPS ultrasound transducer 100. Microbubble/nanodroplet injecting tube 152 may be connected to an infusion pump and to a source of microbubbles and/or nanodroplets. FIG. 4B also illustrates a coaxial cable 154 that is connected to electrical interconnects 126. The center conductor of coaxial cable 154 may be connected to electrical interconnects that are connected to a bonding layer 128 on one axial side of each piezoelectric element 118, and the shield conductor of coaxial cable 154 may be connected to a bonding layer 128 on the opposite axial side of each piezoelectric element 118. Backing layer 122 and acoustic impedance matching layer 124 are also illustrated in FIG. 4B.

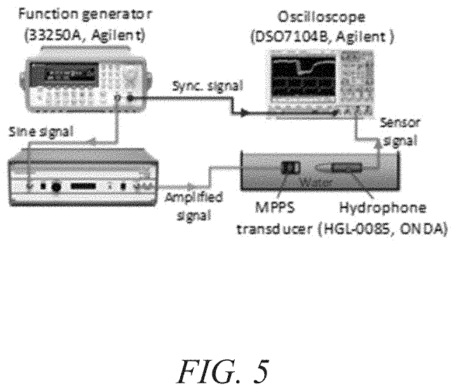

The prototype transducer was characterized based on electric impedance response and acoustic pressure output. Electric impedance response was measured in the frequency range from 5 kHz to 2 MHz using an impedance analyzer (4294A, Agilent Tech. Inc., Santa Clara, Calif.). The impedance curve was compared with the simulation result to confirm the integrity of the fabricated transducer and determine approximate operation frequency condition. The required electric power was estimated using the following formula [19]:

$${P}_{avg} = η \frac{1}{1 - ζ} \frac{{V}_{eff}^{2}}{Z} \qquad ( 3 )$$

where ζ denotes the reflection coefficient due to the electric mismatch, η is the duty cycle (%), Z indicates the electrical impedance at operating frequency of the transducer, and Veff is the effective input voltage. Next, acoustic pressure output induced by the transducer was measured using a hydrophone (HGL-0085, ONDA Corp., Sunnyvale, Calif.). FIG. 5 shows a schematic of the test setup. The function generator (33250A, Agilent Tech. Inc., Santa Clara, Calif.) transmitted a sinusoidal pulse of 15 cycles per 10 ms to the power amplifier (75A250A, AR, Souderton, Pa.). The amplified signal was fed into the MPPS transducer.

### Microbubbles and Nanodroplets Preparation

MBs and NDs were formed by mechanical agitation in accordance with previous research in [32], [41]. The lipid-shelled MBs and NDs were composed of decafluorobutane cores. The concentration of each solution was estimated to be approximately 1×10¹⁰/mL. Each solution was diluted by 10⁻² their original concentration in sterile saline to evaluate the performance of each agent in thrombolysis. Furthermore, the ND solution was diluted by 10⁻³, 10⁻², and 10⁻¹ in saline water, respectively, to investigate the influence of ND concentration. The mean diameters of MBs and NDs particles were 1.1±0.5 μm and approximately 300 nm, respectively [42].

### Blood Clot Incubation

Blood clots for in vitro tests were prepared following our previous works in [23], [28]. Fresh bovine blood (Densco Marketing Inc., Woodstock, Ill.) was mixed with 2.75% (w/v) CaCl₂) solution (Fisher Scientific, Fair Lawn, N.J.) in a volume ratio of 10:1 (i.e., 5 mL CaCl₂) solution for 50 mL bovine blood). The mixture was loaded in Tygon tubes having an inner diameter of 6.35 mm. Next, the Tygon tubes with the blood-CaCl₂) solution were placed inside a 37° C. water bath for three hours to coagulate the blood. The coagulated blood was stored at 4° C. for a week. Finally, the clot samples in the Tygon tubes were sliced into a cylindrical shape to weigh 180 mg±10% in mass.

### Blood Clot Incubation

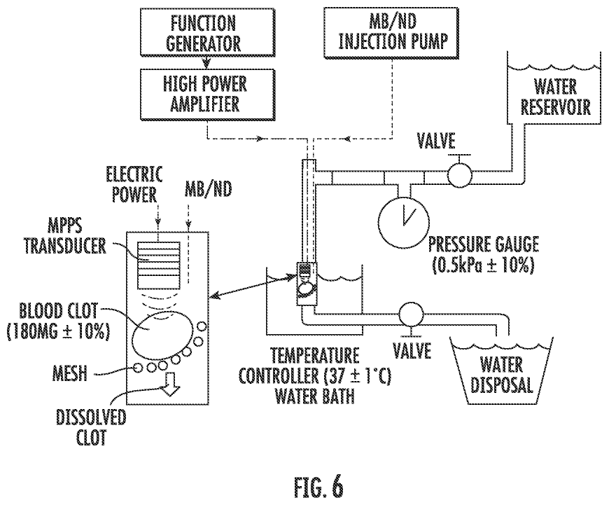

FIG. 6 illustrates a blood flow mimicking system for the demonstration of the thrombolytic efficacy of the developed transducer. A clot sample was placed in the transparent plastic vessel, where a mesh-shape fabric was inserted in the artificial vessel to prevent the blood clot from flowing away while retaining a certain hydraulic pressure level (i.e., 0.5 kPa) [40]. The hydraulic pressure level was controlled in accordance with the height of a water reservoir. The pressure level in the flow system was monitored using a pressure gauge. The US transducer was operated under 80 Vpp electric input and a duty cycle of 8.3% (i.e., 400 pulses for a period, 5 ms). During the 30 min treatment, the clot was continuously insonified for 2 min to activate cavitation of either NDs or MBs and rested for 30 sec to sufficiently disseminate the cavitation agent to the clot.

The test was conducted in both static and dynamic flow models, respectively. For the static flow test, the lysis rate of the ND-mediated sonification (i.e., NDs+US) was compared with other reference cases: MBs+US, US only, and controlled (i.e., without US). Based on the test result in the static flow model, the influence of ND concentration was investigated in the dynamic flow model, varying the ND concentration from 0 to 10⁹ numbers/m L. Each test was repeated three times (n=3).

### Results and Discussion

### Transducer Characterization

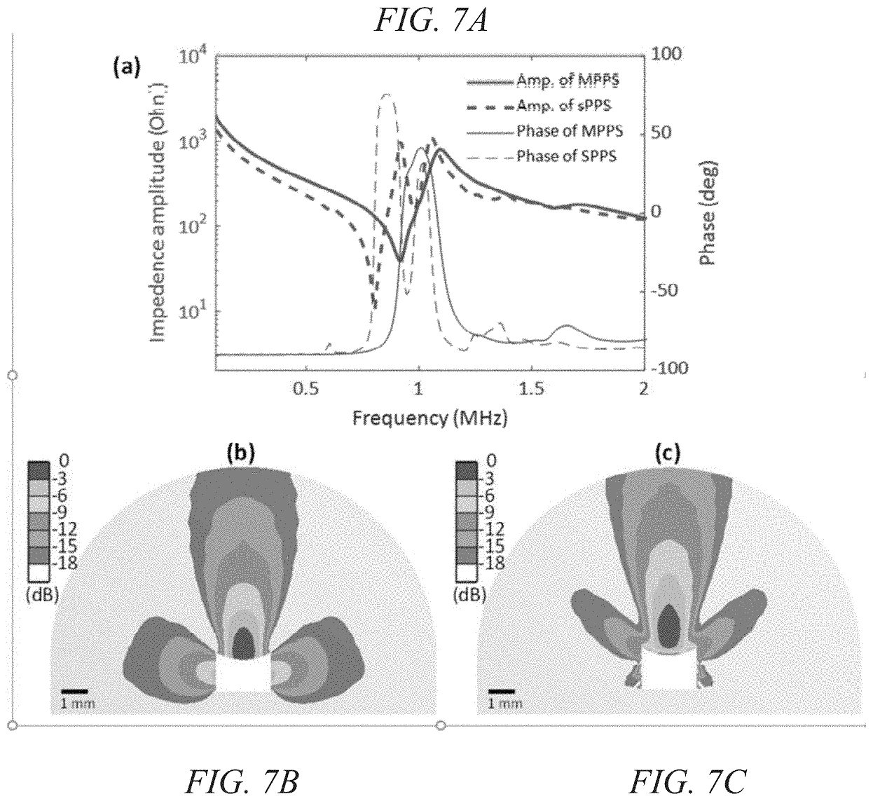

FIG. 7A compares electric impedance responses of the MPPS and the SPPS transducers estimated by the numerical simulation. For the SPPS model, the lateral and the extensional vibration modes are found at 0.80 and 0.98 MHz, respectively. Both modes are slightly coupled together according to the phase diagram. In contrast, in the MPPS model, the lateral vibration mode was almost suppressed owing to the 1-3 composite structure. Meanwhile, the predominant extensional mode was observed at around 0.93 MHz. The MPPS transducer exhibited a relatively broad frequency bandwidth (fᵣ=0.93 MHz and fₐ=1.10 MHz), where fᵣ and fₐ denote the resonance and the anti-resonance frequency, respectively. Furthermore, the impedance amplitude at the resonance (38.9 Ohm) was expected to have a low loss in electric power due to the close electric matching with the electric wire (i.e., 50 Ohm). FIGS. 7B and 7C represent acoustic pressure fields produced by the extensional vibration mode of the SPPS and the MPPS transducer, respectively. While the −6 dB focal zone of the SPPS was from 0 to 1.7 mm from the aperture, that of the MPPS was estimated to be from 0.1 to 3.0 mm. The acoustic beam in the SPPS transducer spreads along the side direction (FIG. 7B), whereas the beam pattern in the MPPS predominantly directs along the forward direction, suppressing acoustic radiation to the side direction (FIG. 7C). For instance, the −12 dB acoustic beam along the side direction reached about 1.33 mm in the SPPS transducer, whereas it was only around 0.56 mm in the MPPS.

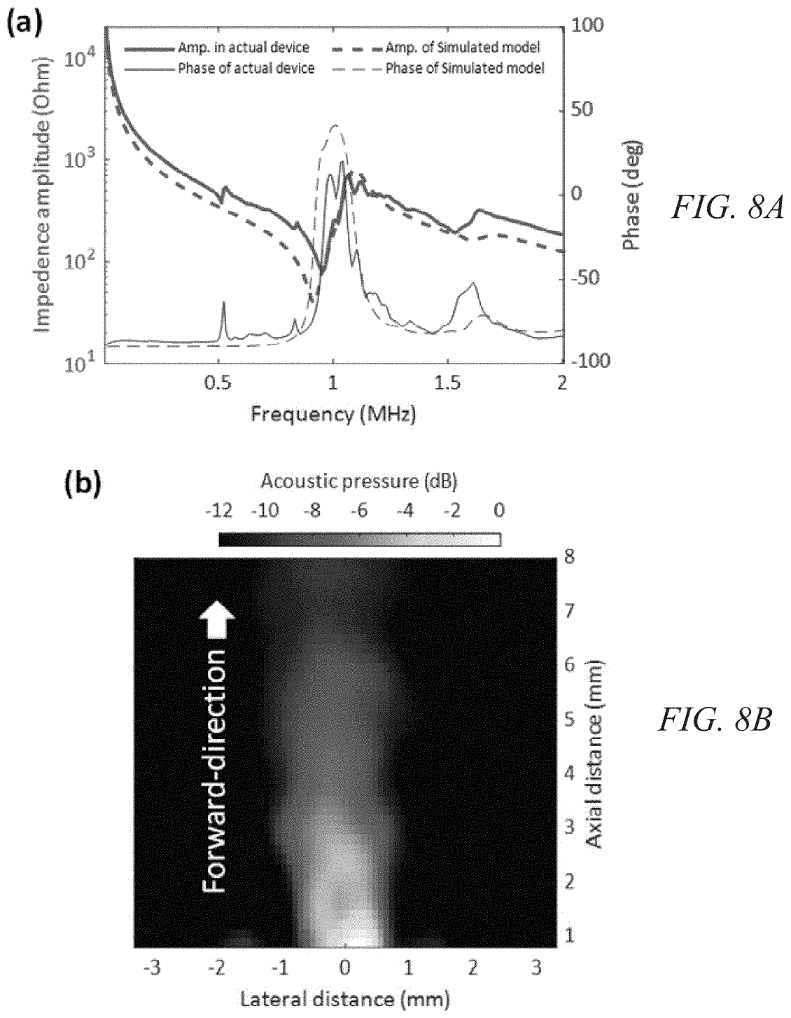

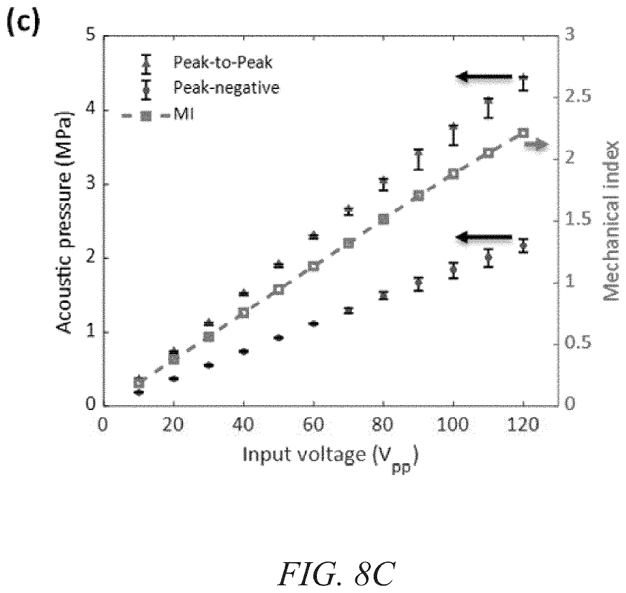

Based on the simulation results, the MPPS transducer was fabricated and characterized as regards electric impedance and acoustic pressure output. FIG. 8A represents the electric impedance of the actual device. The impedance level was about 74.3 Ohms at 0.96 MHz, showing a reliable agreement (˜3.3% discrepancy in the resonance frequency) with the simulation result in FIG. 7A. FIG. 8B is the acoustic pressure field represented in the dB scale. The −6 dB focal zone reached up to 3.4 mm, and the beam width was estimated to be about 1.7 mm. The sensitivity of the transducer was estimated to be about 0.018 MPa/Vₚₚ (FIG. 8C). The corresponding MI was about 1.97 under 120 Vₚₚ input. Since MI level over 1.5 is enough to stimulate both stable and inertial cavitation effects [43], [44], and in vivo application of contrast agents over the level of 1.9 is restricted [45], 80 Vₚₚ was chosen to operate the MPPS transducer in the following in vitro test. MB-mediated cavitation effect induced by an intravascular transducer has been presented and discussed in our previous research in [23]. Cavitation effect of NDs using the intravascular transducer is investigated in further detail below. Table II summarizes the specifications of the MPPS transducer.

TABLE II
SPECIFICATIONS OF THE DEVELOPED
MPPS TRANSDUCER.

| | | | | | |
|---|---|---|---|---|---|
| Operating frequency | 0.96 | MHz | Impedance | 74.3 | Ω |
| −6 dB focal zone | ~3.4 | mm | Beam width | 1.7 | mm |
| Peak-to-peak | 3.05 | MPa | Peak negative | 1.49 | MPa |
| pressure | | | pressure | | |

| | | | | |
|---|---|---|---|---|
| Mechanical index | 1.52 | Electric power¹⁾ | 1.1 | W |

¹⁾under 8.3% duty cycle of 80 Vₚₚ sine actuation

### In Vitro Test Results

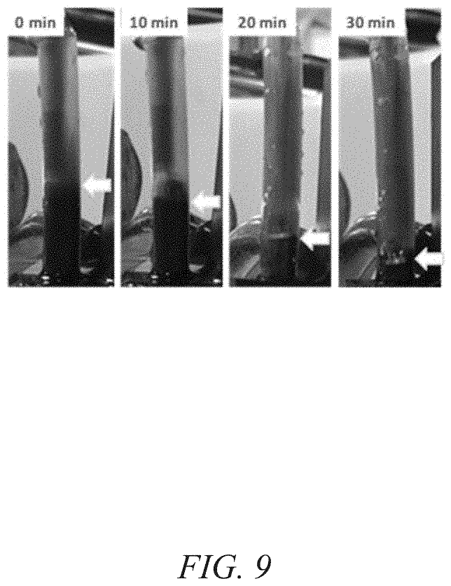

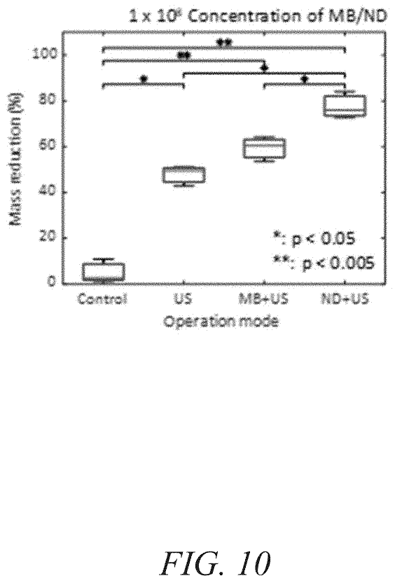

The therapeutic effect of ND-mediated intravascular sonication was initially demonstrated using the MPPS transducer without water flow in the test system. FIG. 9 visualizes the dissolution of a blood clot over time (i.e., 30 min). A red-colored cloud was observed right after operating the transducer due to the cavitation effect in the clot, and the cloud became denser as the clot dissolved further. FIG. 10 compares the lysis rate under different test conditions: NDs+US, MBs+US, US only, and control groups. The ND-mediated ultrasound achieved an average mass reduction of 76.0% and a maximum of 84.1%. The mean lysis rate was estimated at approximately 4.6 mg/min (=2.53%/min). Conversely, percentile mass reductions in the cases of MBs+US and US only were around 60.4% and 49.5%, respectively. The thrombolysis rate obtained by NDs+US is a statistically meaningful improvement compared to other conditions (p<0.05) [46].

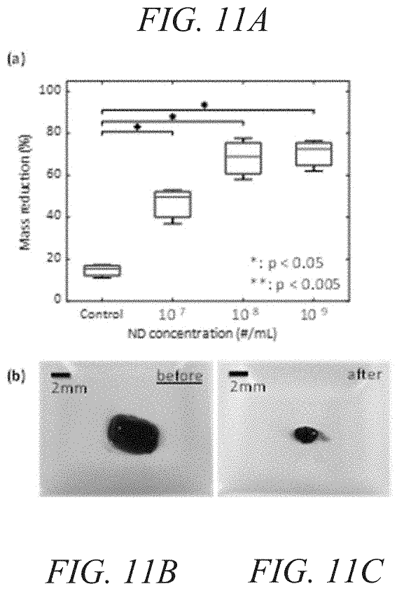

Following the confirmation of the efficacy of ND-mediated sonication, further thrombolysis tests were consequently demonstrated in a flow model (FIG. 6). FIG. 11A presents the thrombolysis rate with respect to ND concentration. While the thrombolysis rate increases in the ND concentration of 10⁸ ND/mL compared with the control and the 10⁷ ND/mL group, there was no significant increase after 10⁹ ND/mL. Notably, the thrombolysis rate in the flow model was slightly decreased by about 9.3% compared to the results without flow. FIGS. 11B and 11C present the bovine blood clot pre- and post-treatment. ND-mediated sonication can successfully dissolve the coagulated blood clot over 76% within 30 min.

### Discussion

An intravascular US transducer was designed to deliver a high acoustic pressure output (>3 MPa in the peak-to-peak level) to a relatively far distance (>3 mm) than the previous forward-viewing intravascular ultrasound designs [23], [28], [29]. The MPPS design demonstrated that the passive material disconnected the lateral connectivity of the active layers, by which the wave transmission along the side direction was effectively suppressed. The simulation results in FIG. 7B affirmed that a typical piezoelectric stack (SPPS) cannot suppress the acoustic pressure output to the side direction due to the predominant lateral vibration mode at 0.8 MHz. In contrast, FIG. 7C exhibited that the predominant extensional vibration mode at about 0.93 MHz is advantageous to deliver the acoustic pressure output along the forward direction (FIG. 8B). For these reasons, the MPPS transducer can be expected to have fewer concerns and clinical complications, such as potential vessel damages caused by unnecessary exposure to the side direction ultrasound beam.

The developed transducer provided a relatively long axial focal zone (>3 mm) (FIG. 8B) compared with existing intravascular devices owing to the MPPS design [23], [28]. The extended focal zone of the MPPS transducer covered the most of an entire clot volume which helped to induce the phase transition (i.e., ND to MB) of NDs and the cavitation within blood clots. The hundreds-nano-sized particles possibly penetrate a blood clot, whereas the typical transducer having either a short focal distance (<1.5 mm) or a low acoustic pressure output (<2 MPa) is not suitable to create sufficient cavitation of ND within the clot. FIG. 10 shows that ND-mediated sonication outperforms other modalities (i.e., US only and US+MBs). The efficient penetration of NDs and the cavitation within the clot can help to disrupt the biostructure more effectively as we anticipated.

The influence of ND concentration was investigated as shown in FIG. 10. The dose over 10⁸ ND/mL did not further increase the mass reduction rate. The distribution of cavitating nanodroplets affects cavitation-induced sonothrombolysis. Although a larger number of cavitating NDs generates more shear-stress in a clot, too many cavitating NDs (i.e., 10⁹ ND/mL) in an ultrasound beam path largely scatters the US energy that hinders the sufficient US delivery for ND cavitation in a further target zone [47]. Meanwhile, compared to the lysis rate without the flow model (FIG. 10), the dissolution rate was reduced by 9.3%. The decrease of the lysis rate in the flow model could be caused as a portion of the injected NDs flows away and does not remain in a static location.

The lysis rate of the proposed method (2.1-2.8%/min) was relatively high in comparison with that of other existing modalities (0.7-1.5%/min), using micron-sized bubble agents combined with intravascular transducers [23], [29], [48]. Such direct comparison would not straightforwardly support the superiority of the proposed method since each study considered the different test parameters in terms of clot size, clot type (e.g., porcine, bovine, and human), and US condition (e.g., duty cycle, voltage level, and frequency). Nonetheless, such a high lysis rate in the ND-mediated intravascular sonication was meaningful as showing the potential of the practical applications. Meanwhile, this study did not consider the influence of chemical agents, such as rt-PA [49]; hence, the application of rt-PA to the ND-mediated sonication can further improve treatment efficacy. Accordingly, the dose of the medications can be minimized by adopting the intravascular thrombolysis technique, thus reducing the possibility of complications, such as bleeding.

The specific aim of this study is a new device development with a preliminary feasibility demonstration. Thus, there is room for further studies as the clinical research on the topic is still in its infantile stages. ND-mediated thrombolysis has to be further validated through either ex vivo or in vivo tests, following in vitro validation using human blood clots as demonstrated in [48]. Histological studies should be also conducted to evaluate the safety of the proposed modality. Parametric studies on the long-term usage of the device should also be encouraged. Moreover, optimal driving conditions (e.g., pulse repetition frequency, duty cycle, driving voltage, and operation frequency) of the device should be further investigated through extensive combinations of the parameters. Nevertheless, the research results presented in this paper demonstrate the clinical potential of the ND-mediated intravascular sonication for DVT treatment.

### CONCLUSION

The subject matter described herein includes a miniaturized, forward-looking, intravascular, ultrasound transducer for the treatment of DVT. The transducer used multi-pillar active elements (similar to a 1-3 composite structure) for piezoelectric stacks and a passive elastomer. Owing to the efficient extensional vibration mode of the transducer, the MPPS transducer can deliver a sufficiently high rarefactional pressure output (˜1.5 MPa) to a far distance (>2λ) from the aperture, where λ is a wavelength of the ultrasound signal produced at an operating frequency of the ultrasound transducer. The acoustic beam produced by the device also exhibited effective directivity along the forward direction, which aided to expedite the ND-mediated thrombolysis. Moreover, compared to common piezoelectric multilayer designs, suppressing the acoustic beam to the side direction potentially would be expected to reduce clinical complications, such as damage in the vessel wall. Meanwhile, the introduction of a flow model degraded treatment efficacy as the NDs could not stay in a static position due to the flow; nonetheless, the percentile mass reduction was still over 68%. Finally, this research result was meaningful in that the relatively high lysis rate (2.1-2.8%/min) was achieved without the aid of thrombolytics. To conclude, the ND-mediated intravascular sonothrombolysis using MPPS transducers will provide an expedited clinical option for DVT treatments.

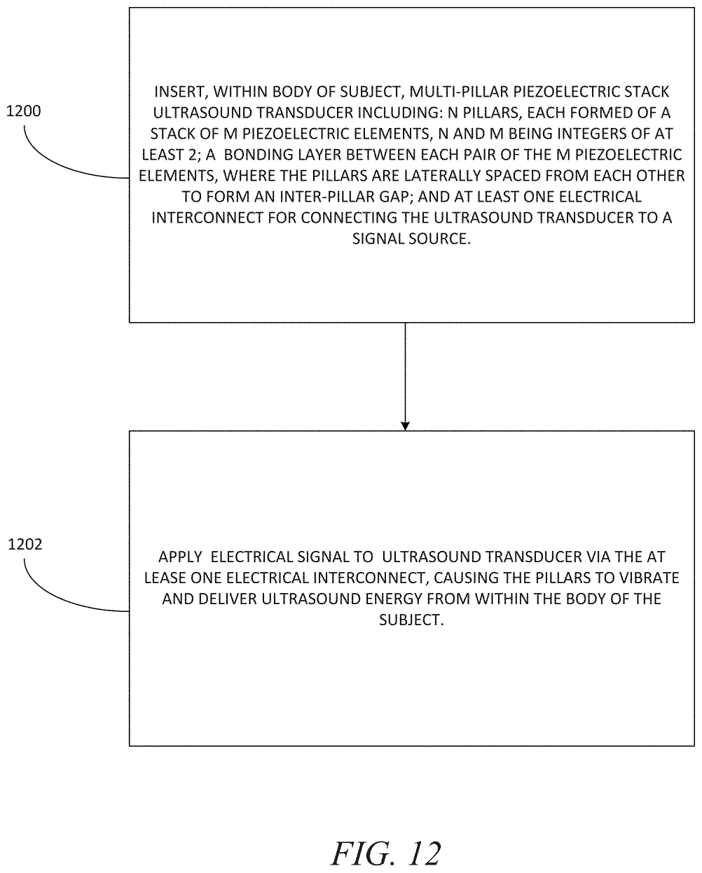

FIG. 12 illustrates an exemplary overall process for delivering ultrasound energy from within a body of a subject using the MPPS transducer described herein. Referring to FIG. 12, in step 1200, the process includes inserting, within the body of the subject, a multi-pillar piezoelectric stack ultrasound transducer including: N pillars, each formed of stacks of M piezoelectric elements, N and M being integers of at least two; a bonding layer between each pair of the M piezoelectric elements, wherein the pillars are laterally spaced from each other to form an inter-pillar gap; and at least one electrical interconnect for connecting the ultrasound transducer to a signal source. For example MPPS transducer 100 illustrated in FIGS. 4A1-4B can be inserted within a blood vessel to deliver ultrasound energy from within the blood vessel.

In step 1202, the process includes applying an electrical signal to the ultrasound transducer via the at least one electrical interconnect, which causes the pillars to vibrate and deliver ultrasound energy from within the body of the subject. For example, the coaxial cable connected to the electrical interconnects on each of the pillars may be connected to a signal source that is configured to generate an electrical signal of a desired frequency and amplitude. The signal source may be activated to apply the electrical signal to the transducer, causing the pillars to vibrate, and delivering ultrasound energy from within the body of the subject. The ultrasound energy may be directed at a target within the body of the subject, such as a blood clot within a blood vessel or other structure outside of a blood vessel within the body of the subject. The frequency and amplitude of the electrical signal may be tailored to the particular application. In one example, the frequency of the electrical signal may be set to a frequency within a range of 100 kHz to 10 MHz.

### Cavitation Effect of Nanodroplets

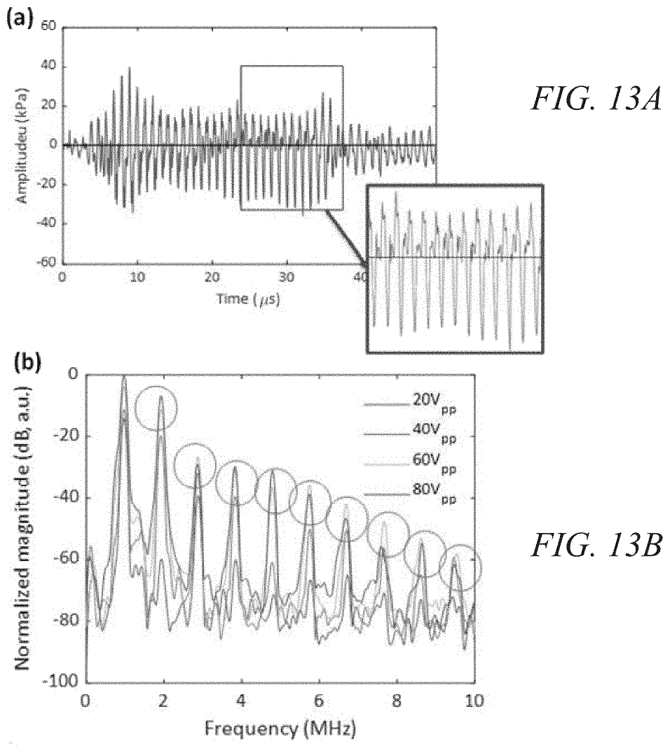

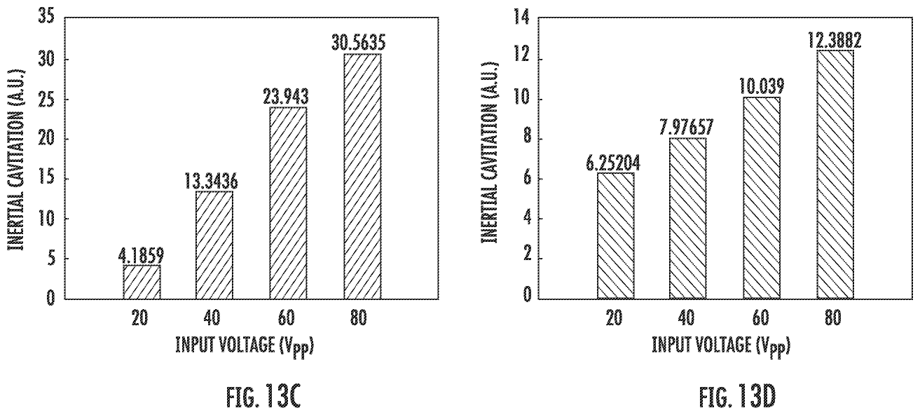

The objective of this section is to confirm the cavitation effect of ND induced by the developed MPPS transducer. The experiment followed our previous setup in [23]. A hydrophone (HGL-0085, ONDA Corp., Sunnyvale, Calif.) measured the acoustic pressure output of the artificial vessel upon the sonication of 30 cycles of a sine wave for 0.5 ms over ND. The measured signal over time was transformed into the frequency domain by using MATLAB (rel. 2019a, MathWorks, Natick, Mass.). To quantify the stable cavitation effect of ND, the frequency signal was filtered in the range of the operation frequency ±10% to obtain the second harmonic of the signal, followed by the summation of the spectrum magnitude. The quantification of the inertial cavitation was obtained by applying the band-stop (i.e., notch) filter to the primary and the super-harmonic frequency bands (marked in the circles in the graph in FIG. 13B) and by summing up the filtered frequency signal. For both the bandpass and the notch digital filtering, the 6th order of Butterworth filter was used.

FIGS. 13A-13D illustrate measurements of the pressure signal measured by the hydrophone. The acoustic pressure output induced by a sinusoidal input (in the inset of FIG. 13A) was significantly distorted due to the shock wave produced by the inertial cavitation and the super-harmonic terms resulting from the stable cavitation of ND. FIG. 13B represents the frequency spectrum of the acoustic pressure signal with respect to the input voltage level of the device. Transmitting a higher acoustic pressure output (i.e., applying a high electric power to the transducer) tends to increase the magnitude of super-harmonics and the broadband noise. FIG. 13C and FIG. 13D quantify the intensity of the stable and the inertial cavitation, respectively. The test results show that the MPPS transducer can generate ND cavitation and increase cavitation effects by amplifying the electric power to the MPPS transducer.

The disclosure of each of the references listed herein is hereby incorporated herein by reference in its entirety.

### REFERENCES

[1] E. Previtali, P. Bucciarelli, S. M. Passamonti, and I. Martinelli, “Risk factors for venous and arterial thrombosis,” Blood Transfusion, vol. 9, no. 2, pp. 120-138, 2011.
[2] S. R. Kahn, “The clinical diagnosis of deep venous thrombosis: integrating incidence, risk factors, and symptoms and signs,” Archives of Internal Medicine, vol. 158, no. 21, pp. 2315-2323, 1998.
[3] F. A. Anderson, H. B. Wheeler, R. J. Goldberg, D. W. Hosmer, N. A. Patwardhan, B. Jovanovic, A. Forcier, and J. E. Dalen, “A population-based perspective of the hospital incidence and case-fatality rates of deep vein thrombosis and pulmonary embolism: the Worcester DVT Study,” Archives of Internal Medicine, vol. 151, no. 5, pp. 933-938, 1991.
[4] M. G. Beckman, W. C. Hooper, S. E. Critchley, and T. L. Ortel, “Venous thromboembolism: a public health concern,” American Journal of Preventive Medicine, vol. 38, no. 4, pp. S495-S501, 2010.
[5] J. P. Galanaud, J. P. Laroche, and M. Righini, “The history and historical treatments of deep vein thrombosis,” Journal of Thrombosis and Haemostasis, vol. 11, no. 3, pp. 402-411, 2013.
[6] H. Robert-Ebadi, G. Le Gal, and M. Righini, “Use of anticoagulants in elderly patients: practical recommendations,” Clinical Interventions in Aging, vol. 4, pp. 165-177, 2009.
[7] L. Mazzolai, V. Aboyans, W. Ageno, G. Agnelli, A. Alatri, R. Bauersachs, M. P. A. Brekelmans, H. R. Büller, A. Elias, D. Farge, S. Konstantinides, G. Palareti, P. Prandoni, M. Righini, A. Torbicki, C. Vlachopoulos, M. Brodmann, “Diagnosis and management of acute deep vein thrombosis: a joint consensus document from the European Society of Cardiology working groups of aorta and peripheral vascular diseases and pulmonary circulation and right ventricular function,” European Heart Journal, vol. 39, no. 47, pp. 4208-4218, 2018.
[8] L. J. Greenfield and M. C. Proctor, “Recurrent thromboembolism in patients with vena cava filters,” Journal of Vascular Surgery, vol. 33, no. 3, pp. 510-514, 2001.
[9] T. G. Van Ha, “Complications of inferior vena caval filters,” Seminars in Interventional Radiology, vol. 23, no. 2, pp. 150-155, 2006.
[10] G. Goudot, T. Mirault, B. Arnal, C. Boisson-Vidal, B. Le Bonniec, P. Gaussem, A. Galloula, M Tanter, E. Messas, and M. Pernot, “Pulsed cavitational therapy using high-frequency ultrasound for the treatment of deep vein thrombosis in an in vitro model of human blood clot,” Physics in Medicine & Biology, vol. 62, no. 24, pp. 9282-9294, 2017.
[11] W. Yang and Y. Zhou, “Effect of pulse repetition frequency of high-intensity focused ultrasound on in vitro thrombolysis,” Ultrasonics Sonochemistry, vol. 35, pp. 152-160, 2017.
[12] R. J. Siegel and H. Luo, “Ultrasound thrombolysis,” Ultrasonics, vol. 48, no. 4, pp. 312-320, 2008.
[13] C. Wright, K. Hynynen, and D. Goertz, “In vitro and in vivo high intensity focused ultrasound thrombolysis,” Investigative Radiology, vol. 47, no. 4, pp. 217, 2012, 2012.
[14] C. J. Shaw, G. R. ter Haar, I. H. Rivens, D. A. Giussani, and C. C. Lees, “Pathophysiological mechanisms of high-intensity focused ultrasound-mediated vascular occlusion and relevance to non-invasive fetal surgery,” Journal of The Royal Society Interface, vol. 11, no. 95, 20140029, 2014.
[15] R. Chopra, N. Baker, V. Choy, A. Boyes, K. Tang, D. Bradwell, and M. J. Bronskill, “MRI compatible transurethral ultrasound system for the treatment of localized prostate cancer using rotational control,” Medical Physics, vol. 35, no. 4, pp. 1346-1357, 2008.
[16] G. Sommer, K. B. Pauly, A. Holbrook, J. Plata, B. Daniel, D. Bouley, H. Gill, P. Prakash, V. Salgaonkar, P. Jones, and C. Diederich, “Applicators for MR-guided ultrasonic ablation of BPH,” Investigative Radiology, vol. 48, no. 6, pp. 387-394, 2013.

[17] D. Melodelima, C. Lafon, F. Prat, Y. Theillère, A. Arefiev, and D. Cathignol, “Transoesophageal ultrasound applicator for sector-based thermal ablation: first in vivo experiments,” Ultrasound in Medicine and Biology, vol. 29, no. 2, pp. 285-291, 2003.
[18] T. D. Mast, P. G. Barthe, I. R. S. Makin, M. H. Slayton, C. P. Karunakaran, M. T. Burgess, and S. M. Rudich, “Treatment of rabbit liver cancer in vivo using miniaturized image-ablate ultrasound arrays,” Ultrasound in Medicine and Biology, vol. 37, no. 10, pp. 1609-1621, 2011.
[19] H. Kim, H. Wu, N. Cho, P. Zhong, K. Mahmood, H. Lyerly, and X. Jiang, “Miniaturized intracavitary forward-looking ultrasound transducer for tissue ablation,” IEEE Transactions on Biomedical Engineering, to be published, 2019.
[20] X. Liu, N. Ellens, E. Williams, E. C. Burdette, P. Karmarkar, C. R. Weiss, D. Kraitchman, and P. A. Bottomley, “High-resolution intravascular MRI-guided perivascular ultrasound ablation,” Magnetic Resonance in Medicine, vol. 83, no. 1, pp. 240-253, 2020.
[21] A. J. Dixon, J. P. Kilroy, A. H. Dhanaliwala, J. L. Chen, L. C. Phillips, M. Ragosta, A. L. Klibanov, and B. R. Wamhoff, and J. A. Hossack, “Microbubble-mediated intravascular ultrasound imaging and drug delivery,” IEEE Transactions on Ultrasonics, Ferroelectrics, and Frequency Control,” vol. 62, no. 9, pp. 1674-1685, 2015.
[22] A. J. Dixon, A. H. Dhanaliwala, J. L. Chen, and J. A. Hossack, “Enhanced intracellular delivery of a model drug using microbubbles produced by a microfluidic device,” Ultrasound in Medicine & Biology, vol. 39, no. 7, pp. 1267-1276, 2013.
[23] J. Kim, B. D. Lindsey, W. Y. Chang, X. Dai, J. M. Stavas, P. A. Dayton, and X. Jiang, “Intravascular forward-looking ultrasound transducers for microbubble-mediated sonothrombolysis,” Scientific Reports, vol. 7, no. 3454, 2017.
[24] U. K. Ballehaninna, A. Hingorani, E. Ascher, A. Shiferson, N. Marks, E. Aboian, R. Jimenez, T. Jacob, and T. McIntyre, “Acute superior mesenteric artery embolism: reperfusion with AngioJet hydrodynamic suction thrombectomy and pharmacologic thrombolysis with the EKOS catheter,” Vascular, vol. 20, no. 3, pp. 166-169, 2012.
[25] FDA News, [Online] https://www.fdanews.com/articles/164759-fda-clears-ekos-ekosonic-endovascular-system, Accessed on May 2020.
[26] C. A. Owens, “Ultrasound-enhanced thrombolysis: EKOS EndoWave infusion catheter system,” Seminars in Interventional Radiology, vol. 25, no. 1, pp. 037-041, 2008.
[27] R. P. Engelberger, D. Spirk, T. Willenberg, A. Alatri, D. D, Do, I. Baumgartner, and N. Kucher, “Ultrasound-assisted versus conventional catheter-directed thrombolysis for acute iliofemoral deep vein thrombosis,” Circulation: Cardiovascular Interventions, vol. 8, no. 1, e002027, 2015.
[28] L. Goel, H. Wu, H. Kim, B. Zhang, J. Kim, P. A. Dayton, Z. Xu, and X. Jiang, “Examining the influence of low-dose tissue plasminogen activator on microbubble-mediated forward-viewing intravascular sonothrombolysis,” Ultrasound in Medicine & Biology, to be published, 2020.
[29] B. Zhang, H. Kim, H. Wu, Y. Gao, and X. Jiang, “Sonothrombolysis with magnetic microbubbles under a rotational magnetic field,” Ultrasonics, vol. 98, pp. 62-71, 2019.
[30] L. Galiuto, L. Badano, K. Fox, R. Sicari, and J. L. Zamorano, The EAE textbook of echocardiography, Ch. 7, Oxford University Press, 2011.
[31] L. Mullin, R. Gessner, J. Kwan, M. Kaya, M. A. Borden, and P. A. Dayton, “Effect of anesthesia carrier gas on in vivo circulation times of ultrasound microbubble contrast agents in rats,” Contrast Media & Molecular Imaging, vol. 6, no. 3, pp. 126-131, 2011.
[32] L. C. Moyer, K. F. Timbie, P. S. Sheeran, R. J. Price, G. W. Miller, and P. A. Dayton, “High-intensity focused ultrasound ablation enhancement in vivo via phase-shift nanodroplets compared to microbubbles,” Journal of Therapeutic Ultrasound, vol. 3, no. 7, 2015.
[33] N. Rapoport, K. H. Nam, R. Gupta, Z. Gao, P. Mohan, A. Payne, N. Todd, X. Liu, T. Kim, J. Shea, C. Scaife, D. L. Parker, E. K. Jeong, and A. M. Kennedy, “Ultrasound-mediated tumor imaging and nanotherapy using drug loaded, block copolymer stabilized perfluorocarbon nanoemulsions,” Journal of Controlled Release, vol. 153, no. 1, pp. 4-15, 2011.
[34] P. C. Chu, W. Y. Chai, C. H. Tsai, S. T. Kang, C. K. Yeh, and H. L. Liu, “Focused ultrasound-induced blood-brain barrier opening: association with mechanical index and cavitation index analyzed by dynamic contrast-enhanced magnetic-resonance imaging,” Scientific Reports, vol. 6, no. 33264, 2016.
[35] T. S. Curry, J. E. Dowdey, and R. C. Murry, Christensen's Physics of Diagnostic Radiology, 4th ed., Lippincott Williams & Wilkins, 1990.
[36] J. Fan, W. A. Stoll, and C. S. Lynch, “Nonlinear constitutive behavior of soft and hard PZT: Experiments and Modeling,” Acta Materialia, vol. 47, no. 17, pp. 4415-4425, 1999.
[37] D. M. Mills and S. W. Smith, “Multi-layered PZT/polymer composites to increase signal-to-noise ratio and resolution for medical ultrasound transducers,” IEEE Transactions on Ultrasonics, Ferroelectrics, and Frequency Control, vol. 46, no. 4, pp. 961-971, 1999.
[38] J. Kim and Y. Roh, “Homogenization of PMN-PT/epoxy 1-3 piezocomposites by resonator measurements and finite element analysis,” Sensors and Actuators A: Physical, vol. 206, pp. 97-106, 2014.
[39] I. D. Johnston, D. K. McCluskey, C. K. L. Tan, and M. C. Tracey, “Mechanical characterization of bulk Sylgard 184 for microfluidics and microengineering,” Journal of Micromechanics and Microengineering, vol. 24, no. 3, 035017, 2014.
[40] J. Ma, S. Guo, D. Wu, X. Geng, and X. Jiang, “Design, fabrication, and characterization of a single-aperture 1.5-MHz/3-MHz dual-frequency HIFU transducer,” IEEE Transactions on Ultrasonics, Ferroelectrics, and Frequency Control, vol. 60, no. 7, pp. 1519-1529, 2013.
[41] P. S. Sheeran, S. H. Luois, L. B. Mullin, T. O. Matsunaga, and P. A. Dayton, “Design of ultrasonically-activatable nanoparticles using low boiling point perfluorocarbons,” Biomaterials, vol. 33, no. 11, pp. 3262-3269, 2012.
[42] J. Kim, R. M. DeRuiter, L. Goel, Z. Xu, X. Jiang, and P. A. Dayton, “A comparison of sonothrombolysis in aged clots between low boiling point phase change nanodroplets and microbubbles of the same composition,” Ultrasound in Medicine and Biology, submitted for publication.
[43] X. Zhang, G. E. Owens, H. S. Gurm, Y. Ding, C. A. Cain, and Z. Xu, “Noninvasive thrombolysis using histotripsy beyond the intrinsic threshold (microtripsy),” IEEE Transactions on Ultrasonics, Ferroelectrics, and Frequency Control, vol. 62, no. 7, pp. 1342-1355, 2015.
[44] J. Šponer, “Theoretical estimation of the cavitation threshold for very short pulses of ultrasound,” Ultrasonics, vol. 29, no. 5, pp. 376-380, 1991.
[45] T. Şen, O. Tüfekçio\check{g}lu, and Y. Koza, “Mechanical index,” Anatolian Journal of Cardiology, vol. 15, no. 4, pp. 334-346, 2015.
[46] S. N. Goodman, “Toward evidence-based medical statistics. 1: The P value fallacy,” Annals of Internal Medicine, vol. 130, no. 12, pp. 995-1004, 1999.
[47] C. Vanhille and K. Hynynen, “Numerical simulations of the nonlinear interaction of a bubble cloud and a high intensity focused ultrasound field,” Acoustics, vol. 1, no. 4, pp. 825-836, 2019.
[48] R. P. Engelberger, V. Schroeder, M. Nagler, R. Prince, D. Poriard, D. Hayoz, and N. Kucher, “Enhanced thrombolysis by ultrasound-assisted catheter-directed thrombolysis and microbubbles in an in vitro model of iliofemoral deep vein thrombosis,” Thrombosis and Haemostasis, vol. 119, no. 7, pp. 1094-1101, 2019.
[49] M. J. Stone, V. Frenkel, S. Dromi, P. Thomas, R. P. Lewis, K. C. P. Li, M. Horne III, and B. J. Wood, “Pulsed-high intensity focused ultrasound enhanced tPA mediated thrombolysis in a novel in vivo clot model, a pilot study,” Thrombosis Research, vol. 121, no. 2, pp. 193-202, 2007.

Many modifications and other aspects of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific aspects disclosed and that equivalents, modifications, and other aspects are intended to be included within the scope of the appended claims. Although specific terms are employed herein, they are used in a generic and descriptive sense only and not for purposes of limitation.

What is claimed is:

1. A multi-pillar piezoelectric stack ultrasound transducer, the ultrasound transducer comprising:
N pillars, each formed of a stack of M piezoelectric elements, N and M being integers of at least two;
a bonding layer between each pair of the M piezoelectric elements;
wherein the pillars are laterally spaced from each other to form an inter-pillar gap; and
at least one electrical interconnect for connecting the ultrasound transducer to a signal source.

2. The ultrasound transducer of claim 1 wherein N is an integer of at least 4.

3. The ultrasound transducer of claim 1 wherein each pillar is greater in axial length than in lateral dimensions.

4. The ultrasound transducer of claim 1 wherein the bonding layer comprises an electrically conductive material and the at least one electrical interconnect is connected to the bonding layer.

5. The ultrasound transducer of claim 4 wherein the at least one electrical interconnect comprises a plurality of electrical interconnects located on lateral faces of the pillars.

6. The ultrasound transducer of claim 1 wherein the piezoelectric elements each have a lateral dimension of more than one wavelength of an ultrasound signal produced at an operating frequency of the ultrasound transducer.

7. The ultrasound transducer of claim 1 comprising at least one of a polydimethylsiloxane (PDMS) material and an epoxy material located in the inter-pillar gap.

8. The ultrasound transducer of claim 1 wherein the piezoelectric elements comprise one of: a lead zirconate titanate material, a lead magnesium niobite-lead titanate material, and a lead-free piezoelectric material.

9. The ultrasound transducer of claim 1 comprising an acoustic impedance matching layer connected to the pillars.

10. The ultrasound transducer of claim 9 wherein the acoustic impedance matching layer comprises one of: an acoustic lens having a concave axially-facing outer surface and a flat aperture.

11. The ultrasound transducer of claim 9 wherein the acoustic impedance matching layer has an acoustic impedance between an acoustic impedance of the piezoelectric elements and an acoustic impedance of an operating medium of the ultrasound transducer.

12. The ultrasound transducer of claim 1 comprising a backing layer connected to the pillars.

13. The ultrasound transducer of claim 12 wherein the backing layer comprises one of: an enclosure that defines an air cavity, a composite with internal air bubbles, and a polymer.

14. The ultrasound transducer of claim 1 wherein the ultrasound transducer achieves a −6 dB focal zone ranging from about zero wavelengths to about two wavelengths from an aperture of the ultrasound transducer, wherein a wavelength is a wavelength of an ultrasound signal defined at an operating frequency of the ultrasound transducer.

15. A system for delivering ultrasound energy within a body of a subject, the system comprising:
a multi-pillar piezoelectric stack ultrasound transducer including:
N pillars, each formed of stacks of M piezoelectric elements, N and M being integers of at least two;
a bonding layer between each pair of the M piezoelectric elements;
wherein the N pillars are laterally spaced from each other to form an inter-pillar gap; and
at least one electrical interconnect for connecting the ultrasound transducer to a signal source; and
a catheter insertable into the body of the subject, wherein the ultrasound transducer is deployable from within the catheter to deliver ultrasound energy from within the body of the subject.

16. A method for delivering ultrasound energy from within a body of a subject, the method comprising:
inserting, within the body of the subject, a multi-pillar piezoelectric stack ultrasound transducer including: N pillars, each formed of stacks of M piezoelectric elements, N and M being integers of at least two; a bonding layer between each pair of the M piezoelectric elements, wherein the pillars are laterally spaced from each other to form an inter-pillar gap; and at least one electrical interconnect for connecting the ultrasound transducer to a signal source; and
applying an electrical signal to the multi-pillar piezoelectric stack ultrasound transducer via the at least one electrical interconnect, which causes the pillars to vibrate and deliver ultrasound energy from within the body of the subject.

17. The method of claim 16 wherein inserting the ultrasound transducer within the body of the subject includes inserting a catheter within the body of the subject and deploying the ultrasound transducer from within the catheter.

18. The method of claim 16 comprising providing an acoustic impedance matching layer on the pillars for acoustic impedance matching between the pillars and an operating medium of the ultrasound transducer.

19. The method of claim 16 wherein applying the electrical signal includes applying the electrical signal having a frequency ranging from 100 kHz to 10 MHz.

20. The method of claim 16 wherein applying the electrical signal to deliver the ultrasound energy includes applying the electrical signal to deliver the ultrasound energy with a −6 dB focal zone ranging from about zero wavelengths to about two wavelengths from an aperture of the ultrasound transducer, wherein a wavelength is a wavelength of an ultrasound signal defined at an operating frequency of the ultrasound transducer.
